(19) United States
(12) Patent Application Publication
(10) Pub. No.: US 2026/0272794 A1
(43) Pub. Date: Sep. 17, 2026

(54) METHODS FOR PREPARING AN EMULSION

(71) Applicant: The Board of Trustees of the University of Illinois, Urbana, IL (US)

(72) Inventors: Sushant Anand, Chicago, IL (US); Rebecca Di Siena, Chicago, IL (US); Pranshi Anand, Chicago, IL (US)

(73) Assignee: The Board of Trustees of the University of Illinois, Urbana, IL (US)

(21) Appl. No.: 19/168,730

(22) PCT Filed: Mar. 19, 2024
(86) PCT No.: PCT/US2024/020564

§ 371 Date: Sep. 24, 2025

Related U.S. Application Data
(60) Provisional application No. 63/454,481, filed on Mar. 24, 2023.

(51) Int. Cl. A61K 8/06; A61K 9/113

(52) U.S. Cl. CPC A61K 8/062; A61K 8/064; A61K 8/066; A61K 9/113; A61K 2800/80

(57) ABSTRACT

Methods and systems for preparing an emulsion are provided herein, which include a multiphase system having an organic phase and an aqueous phase, one of which is frozen and subsequently melted in the liquid phase thereby forming the emulsion. Modulation of temperature, surfactant type and/or concentration, and/or velocity are described for modifying emulsion droplet size.

### INTRODUCTION

This application claims the benefit of priority from U.S. Provisional Application Ser. No. 63/454,481, filed Mar. 24, 2023, the content of which is incorporated herein by reference in its entirety.

This invention was made with government support under grant no. 1847627 awarded by the National Science Foundation. The government has certain rights in this invention.

### BACKGROUND

In physical chemistry, a “colloid” is a homogeneous noncrystalline substance composed of large molecules or ultramicroscopic particles of one substance dispersed through a second substance. Colloids include gels, sols, and emulsions, wherein the particles do not settle, and cannot be separated by filtration or centrifugation. When a colloid is composed of a mixture of two or more liquids that normally are immiscible, an emulsion is formed. In an emulsion, at least one liquid, the dispersed phase, is dispersed as droplets within the other liquid, the continuous phase. The different types of emulsions are dictated by the liquids used in the mix and include oil-in-water (O/W) emulsions and water-in-oil (W/O) emulsions. However, multiple emulsions are also possible and include water-in-oil-in-water (W/O/W) or oil-in-water-in-oil (O/W/O) emulsions.

Emulsions may be stabilized using surfactants, which form films at the surface of the droplets thereby reducing the interfacial tension between phases and creating finer droplets. Surfactants can be soluble in water or oil depending on their hydrophilic-lipophilic balance (HLB) value. Surfactants are known to stabilize emulsions by preventing separation into two liquid layers, Ostwald ripening, aggregation, or phase inversion. The structure of an emulsion may be influenced by factors including, for example, the volume ratio of liquids, the inclusion of a surfactant, the concentration of surfactant used, and temperature.

Emulsions are prepared by emulsification. The most common method is by mechanical means such as magnetic stirring, homogenization, or sonication. These conventional systems and methods of making macroemulsions, microemulsions and nanoemulsions, require the application of shear forces and acoustic vibration. A need exists to develop new methods for preparing emulsions.

### SUMMARY OF THE INVENTION

This invention provides a method for preparing an emulsion by (a) providing a multiphase system having an organic phase and an aqueous phase, wherein the organic phase and/or the aqueous phase comprises at least one surfactant; (b) freezing either the organic phase or the aqueous phase to provide a frozen phase and a liquid phase; and (c) melting the frozen phase in the liquid phase, thereby preparing an emulsion. In some aspects, the multiphase system is an oil-in-water (O/W) emulsion, a water-in-oil (W/O) emulsion, a water-in-oil-in-water (W/O/W) emulsion or an oil-in-water-in-oil (O/W/O) emulsion. In other aspects, the method further includes the step of applying thermal energy or mechanical energy. In some aspects, the method further includes adding to the multiphase system an excipient. According to some aspects, the organic phase includes a water immiscible solvent. In further aspects, the organic phase further includes a co-solvent such as a water miscible organic solvent. In some aspects, one or both of the organic phase or aqueous phase has an active agent therein. In some aspects, the surfactant is a nonionic surfactant such as a polyoxyethylene fatty alcohol ether, polyoxyethylene sorbitan fatty acid ester, polyoxyethylene fatty acid ester, sorbitan ester, glycerol monostearate, polyethylene glycol, polypropylene glycol, cetyl alcohol, cetostearyl alcohol, stearyl alcohol, aryl alkyl polyether alcohol, poloxamer, polaxamine, methylcellulose, hydroxycellulose, hydroxypropylcellulose, hydroxypropylmethylcelIulose, noncrystalline cellulose, polysaccharide, polyvinyl alcohol, polyvinylpyrrolidone, or a combination thereof. In further aspects, the surfactant in the liquid phase is present in an amount of about the critical micelle concentration of the surfactant and/or the liquid phase has a temperature difference of at least 1° C. from the frozen phase. An emulsion prepared by the method of the invention is also provided, as is a cosmetic, pharmaceutical composition and food containing the emulsion.

### BRIEF DESCRIPTION OF THE DRAWINGS

FIGS. 1A-1D. Graphic representation of the experimental setup (FIG. 1A) and evolution of Water-in-Oil emulsion without the use of a surfactant over time (T) (FIG. 1B). Graphic representation of the experimental setup (FIG. 1C) and evolution of Water-in-Oil emulsion over time (T) with the use of SPAN® 80 as surfactant blended in the oil (FIG. 1C). For both experiments, the room temperature was set at 20° C.

FIG. 2. Detailed time evolution of droplet formation in a Water-in-Oil emulsion prepared with 10 mM SPAN® 80 at room temperature 20° C. Arrows show daughter droplets.

FIG. 3. Scatter plot of pinch-off time and diameter with surface concentration variation.

FIG. 4. A graph showing the size of dispersed drops obtained when pure ice melts at different temperatures inside a solution of kerosene and SPAN® 80.

FIG. 5. An example emulsion prepared by the method of the invention showing dimpled drops indicative of complex emulsions (oil-in-water-in oil) type emulsions.

FIG. 6. Schematic depiction of a system of the invention.

### DETAILED DESCRIPTION OF THE INVENTION

Conventional emulsion-making techniques use shear force or acoustic cavitation to disrupt the dispersed phase into smaller droplets. The emulsions of this invention do not require the use of any shear forces and instead rely on thermodynamic status, hydrodynamic properties, gravity effect, and interfacial characteristics of both phases. In particular, this invention is a method for preparing emulsions by (a) providing a multiphase system having an organic phase and an aqueous phase, wherein the organic phase and/or the aqueous phase includes at least one surfactant; (b) freezing either the organic phase or the aqueous phase to provide a frozen phase and a liquid phase; and (c) melting the frozen phase in the liquid phase to prepare the emulsion. Advantageously, the method of the invention reduces energy requirements and equipment costs compared to conventional methods for producing emulsions. The emulsions and methods herein find application in a number of different industries including the food, agrochemical, cosmetic, personal care, home cleaning, and pharmaceutical industries.

The term “multiphase system” refers to a dispersion having at least one organic phase and at least one aqueous phase. In one aspect of the invention, the multiphase system is in the form of a reverse emulsion or water-in-oil emulsion (W/O), where the oil phase forms the continuous phase and water the dispersed phase. In another aspect of the invention, the multiphase system is in the form of an oil-in-water emulsion (0/W), where the water phase forms the continuous phase, and the oil phase forms the dispersed phase. The present invention further contemplates emulsions having more than two phases such as an oil-in-water-in-oil emulsion (O/W/O) or a water-in-oil-in-water emulsion (W/O/W). The present invention is a liquid-in-liquid dispersion multiphase system. In some aspects, the multiphase system is a liquid-in-liquid dispersion in the form of an oil-in-water emulsion.

In some aspects, the oil-in-water emulsion is prepared by melting a frozen organic phase (oil) in an aqueous phase (water). In some aspects, the multiphase system is a liquid-in-liquid dispersion in the form of a water-in-oil emulsion.

In some aspects, the water-in-oil emulsion is prepared by melting a frozen aqueous phase (ice) in an organic phase (oil).

In some aspects, the mean diameter of the droplets of the emulsion are of a size in the range of 1 mm to 1000 mm (e.g., 1, 2, 3, 4, 5, 6, 7, 8, 9, 10, 20, 30, 40, 50, 60, 70, 80, 90, 100, 200, 300, 400, 500, 600, 700, 800, 900, 1000 mm, or any range therein). In other aspects, the mean diameter of the droplets of the emulsion are of a size in the range of 1 μm to 1000 μm (e.g., 1, 2, 3, 4, 5, 6, 7, 8, 9, 10, 20, 30, 40, 50, 60, 70, 80, 90, 100, 200, 300, 400, 500, 600, 700, 800, 900, 1000 μm, or any range therein). In a further aspect, the mean diameter of the droplets of the emulsion are of a size in the range of 1 nm to 1000 nm (e.g., 1, 2, 3, 4, 5, 6, 7, 8, 9, 10, 20, 30, 40, 50, 60, 70, 80, 90, 100, 200, 300, 400, 500, 600, 700, 800, 900, 1000 nm, or any range therein), In some aspects, the mean diameter of the droplets of the emulsion are of a size in the range 500 μm to 10 mm (e.g., 500 μm, 600 μm, 700 μm, 800 μm, 900 μm, 1 mm, 2 mm, 3 mm, 4 mm, 5 mm, 6 mm, 7 mm, 8 mm, 9 mm, 10 mm, or any range therein). In accordance with the principles herein, the size of emulsion droplets can be obtained and controlled through varying the parameters of the method, including surfactant concentration, surfactant type, temperature difference between solidified first liquid solution and the bulk second liquid solution.

The organic phase of the multiphase system is preferably a water immiscible or a partially water miscible organic solvent. As used herein, the term “immiscible” means that the organic solvent has a solubility in the aqueous solution of less than about 10 wt %, less than about 5 wt %, or even less than about 3 wt %. In one aspect, the organic solvent is also volatile with a boiling point of 150° C. or less. In some aspects, the water immiscible solvent is a partially water miscible solvent, which is water immiscible at one concentration, and water miscible at another lower concentration. Suitable water immiscible solvents include, but are not limited to, substituted or unsubstituted, linear, branched or cyclic alkanes with a carbon number of 5 or higher; substituted or unsubstituted, linear, branched or cyclic alkenes with a carbon number of 5 or higher; substituted or unsubstituted, linear, branched or cyclic alkynes with a carbon number of 5 or higher; aromatic hydrocarbons; completely or partially halogenated hydrocarbons; ethers; esters; ketones; mono-, di- or tri-glycerides; native oils; alcohols; aldehydes; acids; amines; linear or cyclic silicones; hexamethyldisiloxane; or any combination of these solvents. Halogenated solvents include, but are not limited to carbon tetrachloride, methylene chloride, chloroform, tetrachloroethylene, trichloroethylene, trichloroethane, hydrofluorocarbons, chlorinated benzene (mono, di, tri), trichlorofluoromethane.

Exemplary solvents are methylene chloride, chloroform, diethyl ether, toluene, xylene, kerosene, native oils (e.g., a vegetable oil, a nut oil, or mineral oil), and ethyl acetate. These solvents are of limited water miscibility and capable of spontaneous emulsion formation. Examples of partially water miscible solvents are tetrahydrofuran (THF), propylene carbonate, benzyl alcohol, and ethyl acetate.

In some aspects, the organic phase may also include one or more co-solvents. In some aspects, the co-solvent is a water miscible organic solvent, such as ethanol, methanol, butanol, or acetone.

The ratio by weights of the organic phase to the aqueous phase is from about 1:99 to about 99:1, more preferably from 30:70 to about 70:30, and most preferably from about 40:60 to about 60:40, or any range or combination of ranges therein.

In accordance with the method and emulsion of this invention, the organic phase and/or the aqueous phase includes at least one surfactant. A “surfactant” or “surface active compound” is a substance that, when added to a liquid, reduces its surface tension, thereby increasing its spreading and wetting properties. The surfactant of this invention may be an anionic surfactant, a cationic surfactant, a zwitterionic surfactant, a nonionic surfactant, a biological surface-active molecule, or a combination thereof. The surfactant may also include a polymeric surfactant, nanoparticles (Pickering) surfactants, or a combination of multiple surfactant types. One or more surfactants (e.g., one, two, three, four, five, six, seven, eight, nine, ten or more) may be added to the organic phase, the aqueous phase or to both the organic phase and the aqueous phase. In aspects where both the organic phase and the aqueous phase include at least one surfactant, the surfactant of the organic phase and the surfactant of the aqueous phase are different. In some aspects, the organic phase includes at least a first nonionic surfactant and the aqueous phase includes at least a second nonionic surfactant, wherein the at least first and at least second nonionic surfactants are different. In some aspects, the surfactant is in the frozen phase. In some aspects, the surfactant is in the liquid phase. In some aspects, a surfactant is present in both the frozen phase and the liquid phase. In aspects where both the frozen phase and the liquid phase include at least one surfactant, the surfactant of the liquid phase and the surfactant of the frozen phase are different. In some aspects, the liquid phase includes a first nonionic surfactant, and the frozen phase includes a second nonionic surfactant, wherein the first and second nonionic surfactants are different. The surfactant may be present in an amount by weight of the aqueous phase or the organic phase, whatever the case may be, from about 0.001% to about 10%, more preferably from about 0.01% to about 5%, or any range or combination of ranges therein.

Suitable anionic surfactants include, but are not limited to, potassium laurate, sodium lauryl sulfate, sodium dodecylsulfate, alkyl polyoxyethylene sulfates, sodium alginate, dioctyl sodium sulfosuccinate, phosphatidyl choline, phosphatidyl glycerol, phosphatidyl inosine, phosphatidylserine, phosphatidic acid and their salts, glyceryl esters, sodium carboxymethylcellulose, cholic acid and other bile acids (e.g., cholic acid, deoxycholic acid, glycocholic acid, taurocholic acid, glycodeoxycholic acid) and salts thereof (e.g., sodium deoxycholate, etc.).

Suitable cationic surfactants include, but are not limited to, quaternary ammonium compounds, such as benzalkonium chloride, cetyltrimethylammonium bromide, lauryldimethylbenzylammonium chloride, acyl carnitine hydrochlorides, or alkyl pyridinium halides. As anionic surfactants, phospholipids may be used. Suitable phospholipids include, for example, phosphatidylcholine, phosphatidylethanolamine, phosphatidylserine, phosphatidyl inositol, phosphatidylglycerol, phosphatidic acid, lysophospholipids, egg or soybean phospholipid or a combination thereof. The phospholipid may be salted or desalted, hydrogenated, or partially hydrogenated or natural, semisynthetic or synthetic.

Suitable nonionic surfactants include: polyoxyethylene fatty alcohol ethers (Macrogol and BRIJ™), polyoxyethylene sorbitan fatty acid esters (Polysorbates), polyoxyethylene fatty acid esters, sorbitan esters (SPAN®), glycerol monostearate, polyethylene glycols, polypropylene glycols, cetyl alcohol, cetostearyl alcohol, stearyl alcohol, aryl alkyl polyether alcohols, polyoxyethylene-polyoxypropylene copolymers (poloxomers), polaxamines, methylcellulose, hydroxycellulose, hydroxy propylcellulose, hydroxy propylmethylcellulose, noncrystalline cellulose, polysaccharides including starch and starch derivatives such as hydroxyethylstarch (HES), polyvinyl alcohol, and polyvinylpyrrolidone. In some aspects of the invention, the nonionic surfactant is a sorbitan monooleate. Such polymers are sold under the tradename SPAN® by several suppliers including Sigma-Aldrich. Among the polyoxyethylene glycols is polyethylene glycol sorbitan monolaurate. One example of such a surfactant is TWEEN® 20, sold by Croda.

Surface active biological molecules include such molecules as albumin, casein, heparin, hirudin, hetastarch or other appropriate biocompatible agents.

In some aspects, the multiphase system further includes the addition of at least one excipient. In some aspects, the excipient is added to the organic phase, the aqueous phase or to both the organic phase and the aqueous phase. In aspects where both the organic phase and the aqueous phase include at least one excipient, the excipient of the organic phase and the excipient of the aqueous phase may be the same or different. In some aspects, the excipient may be a protein, salt (e.g., sodium chloride), sugar (e.g., a monosaccharide such as glucose or disaccharide such as sucrose), or sugar alcohol (e.g., mannitol or sorbitol). In aspects where the excipient is a protein, the protein may function as both an excipient and a surfactant.

The multiphase system may further include at least one active agent (e.g., one, two, three, four, five, six, seven, eight, nine, ten or more active agents). An “active agent” refers to any compound that has agronomic, cosmetic, personal care, cleaning, nutraceutical and/or therapeutic effect. The active agent may be in the form of a liquid or solid (e.g., solid particles such as solid nanoparticles or solid microparticles). The active agent may be highly water soluble (i.e., greater than 100 mg/mL), sparingly water soluble (i.e., 5-30 mg/mL), or poorly water soluble (i.e., less than 5 mg/mL). In one aspect, the active agent is “poorly water soluble,” and the active agent has a solubility in water (over the pH range of 6.5 to 7.5 at 25° C.) of less than 5 mg/mL. The active agent may have an even lower aqueous solubility, such as less than about 1 mg/mL, less than about 0.1 mg/mL, and even less than about 0.01 mg/mL. In some aspects, the active agent is added to the organic phase, the aqueous phase or to both the organic phase and the aqueous phase. In aspects where both the organic phase and the aqueous phase include at least one active agent, the active agent of the organic phase and the active agent of the aqueous phase may be the same or different.

Particularly suitable active agents include pharmaceuticals, food or food additives, pesticides, herbicides, fungicides, or cosmeceuticals. Examples of such active agents include, but are not limited to, antihyperlipidemics, anesthetics, anti-asthmatics, antimicrobials (e.g., sulfadiazine), antifungals (e.g., itraconazole), antineoplastics, non-steroidal anti-inflammatory drugs (e.g., indomethacin), anti-hypercholesteremic agents (e.g., probucol), analgesics, steroidal compounds (e.g., dexamethasone), antipyretics, antidepressants, anti-arrhythmics, antianxiety drugs, antimanics, antiarthritics, antihistamines, anti-infectives, water insoluble vitamins, antipsychotics, sedatives, antihypertensive agents, diagnostic agents, anticonvulsants and/or immunosuppressant (e.g., cyclosporin A, tacrolimus, and mycophenolate mofetil). The active agent may be a protein, antibody, antibody fragment, peptide, oligonucleotide, vaccine, or derivative of such materials. In some aspects, the active agent is a small molecule, e.g., a molecule having a molecular weight of about 2000 Daltons or less. The active agent may be present in a concentration to the extent it is soluble in the aqueous phase or organic phase. In some aspects, the active agent may be present in an amount from less than 1% to about 40%, more preferably from about 1% to about 25%, and most preferably from about 1% to about 10% by weight of the organic phase or aqueous phase, or any range or combination of ranges therein.

As indicated herein, the emulsion of the invention is prepared by freezing either the organic phase or the aqueous phase and melting the frozen phase (i.e., frozen organic phase or frozen aqueous phase) in the liquid phase (i.e., liquid organic phase or liquid aqueous phase). The frozen phase may be prepared by any suitable technique and may include one or more freezing molds or containers, and the use of a freezer, dry ice, or other suitable freezing mechanism. Any suitable volume of organic phase and aqueous phase may be used without limitation.

The emulsion of this invention is formed by melting the frozen phase in the liquid phase. Initial and daughter droplet size may be controlled by a number of factors including the surfactant concentration, surfactant type, and temperature difference between the frozen phase and liquid phase. In some aspects, the surfactant in the liquid phase is present in an amount of about the critical micelle concentration (CMC) of the surfactant, e.g., within 0.5% to about 10% of the CMC. In other aspects, the surfactant in the liquid phase is present in an amount above the CMC of the surfactant. Tn other aspects, the surfactant in the liquid phase is present in an amount below the CMC of the surfactant. The CMC is defined as the concentration of surfactants above which micelles form and all additional added surfactants go to micelles. CMC may be determined in water (without other additives or salts) at 25° C. under atmospheric pressure. The CMC of surfactants are generally known in the art and several examples are presented in Table 1.

TABLE 1

| Surfactant | CMC | Category |
|---|---|---|
| Sodium octyl sulfate | 0.13M | anionic |
| Sodium dodecyl sulfate | 0.0083M | anionic |
| Sodium tetradecyl sulfate | 0.0021M | anionic |
| Decyltrimethylammonium bromide | 0.065 | cationic |
| TWEEN ® 20 | 0.06 mM | nonionic |
| SPAN ® 80 | 0.1 mM | nonionic |

In some aspects, the liquid phase has a temperature difference of at least 1° C. from the frozen phase. In other aspects, the temperature difference between the frozen phase and liquid phase is at least 1° C., e.g., at least about 1, 2, 3, 4, 5, 6, 7, 8, 9, 10, 20, 30, 40, 50, 60, 70, 80, 90, or 100° C., or any range therein. In some aspects, the temperature of the frozen phase is at the freezing point (T_{fp}) and the temperature of the liquid phase is greater than the T_{fp} by at least 1° C., e.g., at least about 1, 2, 3, 4, 5, 6, 7, 8, 9, 10, 20, 30, 40, 50, 60, 70, 80, 90, or 100° C., or any range therein. As will be appreciated by those of ordinary skill in the art, the T_{fp} of any given liquid is dependent upon the characteristics of that liquid. For example, the T_{fp} of water is 0° C. and the T_{fp} of peanut oil is 3° C. Freezing points of any given liquid are well-known in the art and/or readily determined by conventional methods. In some aspects, the temperature difference between the frozen phase and liquid phase is in the range of about 1° C. to about 60° C., preferably about 5° C. to about 20° C. The temperature of the liquid phase may be monitored by a sensor or thermometer and increased by the addition of thermal energy (heat) to the liquid phase. In some aspects, the liquid phase provides thermal energy for the frozen phase to melt. The liquid phase itself may be heated from an outside source thereby that energy to the frozen phase to melt it. In some aspects, the upper limit of heating is the boiling point of the liquid phase.

Emulsification can be further facilitated by the addition of mechanical energy to the multiphase system so that thinner melt films are created around the frozen phase, which will then undergo pinch-off to produce finer dispersions. In particular, the liquid phase may be shaken, agitated, vortexed, mixed, or stirred to modify the droplet size of the emulsion. In some aspects, an increase in the rate of shaking, agitating, vortexing, mixing, or stirring may be used to decrease the size of the emulsion droplets until a desirable droplet size is obtained.

The invention also provides an emulsion prepared by the method of this invention. The emulsion resulting from the method may be in the form of a fine dispersion, where one liquid is dispersed into a second liquid (bulk phase). Such fine dispersion systems may or may not be thermally stable. During the formation of the emulsion, solid particles may be formed. These solid particles may be dispersed in the organic phase and/or the aqueous phase of the multiphase system. The solid particles may remain in the emulsion or be removed. Furthermore, the emulsion may be filter sterilized, e.g., using a 0.22 μm membrane filter.

The emulsion of this invention may be used in a wide variety of applications such as in drug delivery, e.g., delivery of medicines or vaccines; in the preparation of films or pastes; in the food industry, e.g., in the preparation of micro-emulsions used in beverages, deserts, and the like; in cosmetics such as hand sprays, hand creams and lotions; in the delivery of pesticides, insecticides, fungicides in agrochemicals; and in industrial applications for oil recovery and water treatment.

The invention further provides a system for preparing the emulsion of the invention. The system 100 is generally depicted in FIG. 6 and includes a mixing chamber 110 having temperature regulating 120 and/or stirring 130 components therein. The mixing can be automated via a suitable processor 140 connected directly or indirectly to the system 100. Alternatively, the processor 140 may be integrated into the mixing chamber 110, if desired. The system 100 can include delivery components 150 for delivering the aqueous phase and/or organic phase or components thereof to the mixing chamber 110. In some aspects, the mixing chamber 110 and processor 140 can be configured to facilitate the selection of aqueous phase and/or organic phase or components thereof and temperatures to achieve a variety of processes.

The processor 140 of the system 100 may be configured to perform melting processes of the frozen phase in accordance with the method herein and can be programmed to execute temperature regulation steps via a temperature regulating component 120. The temperature regulating component 120 can be configured to regulate the melting processes, where a frozen phase melts in liquid phase and the melt detaches from the frozen phase and disperses in the liquid phase.

When used in conjunction with the method of this invention, a frozen phase (e.g., the organic phase or aqueous phase frozen below its freezing point) and liquid phase (at a temperature greater than the freezing point of the frozen phase) are provided to the mixing chamber 110 of the system 100. The processor 140 provides executable instructions to the mixing chamber 110 to begin melting of the frozen phase into the liquid phase and controlling one or more aspects of the liquid phase to modify the properties of the emulsion.

An exemplary method of modifying the properties of the emulsion may include controlling the dimensions of daughter drops by adjusting the concentration of a surfactant via a delivery components 150. Another exemplary method of modifying the properties of the emulsion may include controlling dimensions of daughter drops by adjusting the temperature difference between the frozen phase and the liquid phase via temperature regulating component 120. Still another exemplary method of modifying the properties of the emulsion may include controlling dimensions of daughter drops by adjusting the velocity of a melt-film and a surrounding liquid phase, wherein adjusting the velocity comprises stirring at least one of the frozen phase and/or the liquid phase via stirring component 130.

Unlike conventional systems and methods which use shear forces or acoustic cavitation, produced by a pump injector or a rotor-stator system or an ultrasonic machine, the system and method herein facilitates the preparation of emulsions without using any shear stress induced by external flow. Not only do the systems and methods herein eliminate the need to use any shear forces, but also processes herein may be carried out spontaneously by means of thermodynamic status, hydrodynamic properties, gravity effect and interfacial characteristics of both dispersed and continuous phases.

The following non-limiting examples are provided to further illustrate the present invention.

### Example 1: Overview of Emulsion Preparation

Exemplary steps to make Liquid1 drops inside a bulk Liquid2.

Step 1: Prepare a solution of Liquid1+(M)ₓ+(S)_{y}, wherein “M” is a molecule that is soluble in Liquid1 and is to be delivered via emulsion and “x” is ≥1; and “S” is a surfactant or co-surfactant that is soluble in Liquid1 and “y” is ≥0. The surfactants may or may not be included to decrease the surface tension of the Liquid1-Liquid2 interface. As an example, Liquid1 solution may be water+Salt (M₁)+water soluble drug (M₂)+TRITON™ X-100 (S₁).

Step 2: Prepare a solution of Liquid2+(M)ₓ+(S)_{y}, wherein “M” is a molecule that is soluble in Liquid2 and “x” is ≥1; and “S” is a surfactant or co-surfactant that is soluble in Liquid2 and “y” is ≥0.

Step 3: Freeze the Liquid1 solution below its freezing point (T_{fp}).

Step 4: Immerse solidified Liquid1 in Liquid2 solution. Liquid2 solution should be at a temperature >T_{fp}.

Step 5: As Liquid1 melts in Liquid2, small drops of Liquid1 will self-detach and disperse in Liquid2. To decrease the size of droplet formed further, the operator may:
(i) decrease the Liquid2 solution to be just over T_{fp}; and/or
(ii) add stirring action. This may be done either by rotating the solidified Liquid1 solution, or by stirring Liquid2. The stirring rate may be increased to decrease the size of the droplet until a desirable droplet size is obtained.

In an embodiment, drug molecules to be dispersed through emulsion drops may be first dissolved in the Liquid1. Liquid1 solution (with drug molecules) may then be frozen and subsequently immersed in a bath of Liquid2 (containing surfactants and other molecules). In another embodiment, multiple freezing molds may be prepared and all immersed in the Liquid2 bath together or sequentially. In still another embodiment, a flowing Liquid1 solution may be frozen at the end of a tube, for example, and then the frozen material may be dropped/dispersed in Liquid2 bath. In yet another embodiment, an emulsion may be formed by dispersing Liquid1 solution and Liquid2 solution, freezing the dispersion, and then combining the solid dispersion with Liquid1 or Liquid3, wherein each of Liquid1, Liquid2 and/or Liquid3 containing the same or different drug molecules and surfactants.

In order to maintain consistent mixing ratios in industrial processes at a large scale, a number of embodiments are contemplated. For example, in an embodiment, solidified Liquid1 solution may be affixed to the end of an arm, said end being submerged in Liquid2, wherein the arm is used to stir Liquid2 at a fixed speed. In another embodiment, the Liquid2 bath may be stirred manually or automatically with the help of arms, magnetic stirrers, or other suitable mixing components.

### Example 2: Water-In-Oil (W/O) Emulsion Preparation

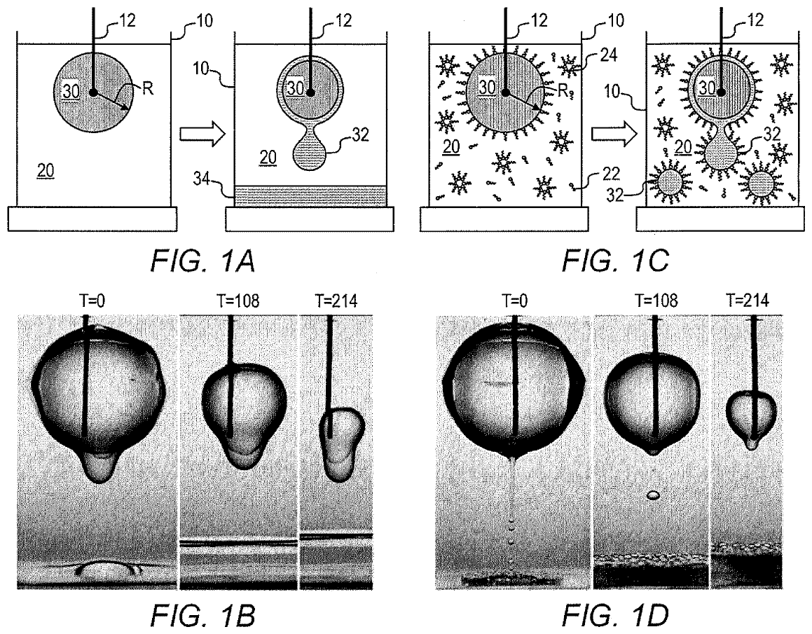

FIG. 1A and FIG. 1C show the basic experimental setup. A beaker 10 was used to hold the continuous phase 20. For these experiments, the continuous phase 20 was kerosene, without (FIG. 1A) or with surfactant 22 (FIG. 1C) (e.g., SPAN® 80 (Sorbitan monooleate)). The continuous phase 20 was kept at ambient temperature and the dispersed phase (water) was frozen into a sphere 30. A wire 12 was used to keep the transparent ice sphere 30 from falling down due to gravity. As the ice sphere 30 melted, droplets 32 were formed.

In the absence of a surfactant 22 (FIG. 1A-1B), the daughter drops 32 were relatively large, creating a sediment 34 at the bottom of the container. By comparison, the droplets 32 of the dispersed phase (water) were dispersed in the continuous phase oil 20 (Kerosene) in presence of a surfactant 22 that adsorbs at the oil-water interface to stabilize a single emulsion 40 (FIG. 1C-ID). In this method the water droplets were not generated by injecting mechanical energy into the system, they were formed by melting. The minimum surfactant concentration required to stabilize the emulsion was adjusted by CMC (critical micelle concentration), whereas for values less than CMC, the surfactants migrate to interfaces and above that accumulate together and form a micelle 24.

By increasing the surfactant concentration, the interfacial between the phases declines significantly until it reaches a CMC concentration that remains unchanged afterward. For the surfactants tested, the concentration used was above the CMC to ensure the complete coverage of emulsion droplets thereby causing a delay of immediate coalescence between droplets upon contact.

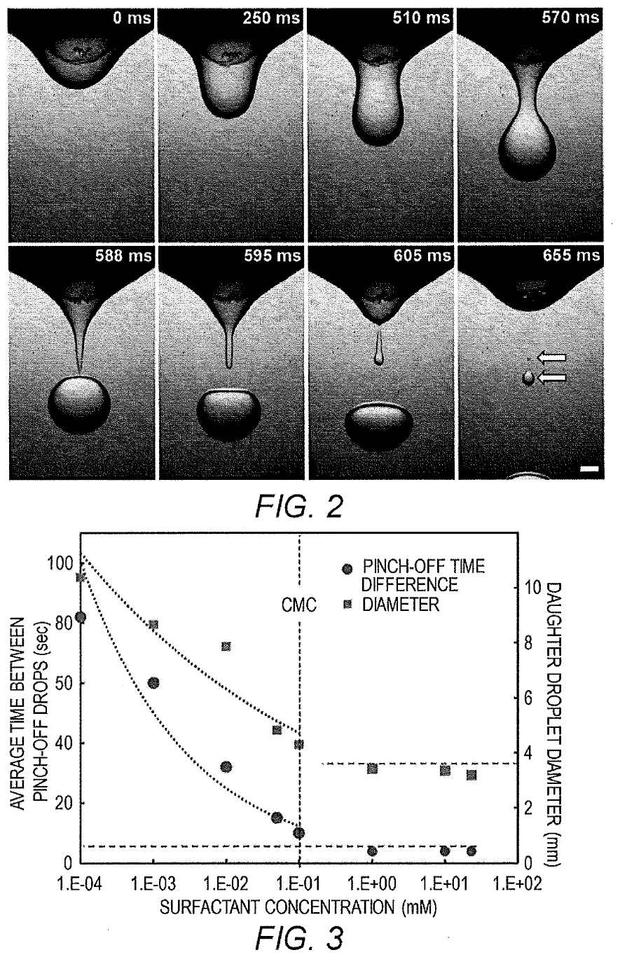

When adding 10 mM of SPAN® 80 to kerosene, the Plateau-Rayleigh instability lead to the formation firstly of a continuous downward flow of water, being denser than oil, followed subsequently by a stable phase in which it was possible to observe the formation of a larger droplet followed by two smaller daughters. A detailed image of the above-mentioned instances is presented in FIG. 2. As shown in FIG. 1D, it is possible to observe a layer of water spheres protected by the surfactant thereby avoiding coalescence.

Another relevant aspect is the link between droplet size, surface concentration, and pinch-off time. Upon increasing the amount of SPAN® 80 to the CMC value (0.1 mM for SPAN® 80), both the dimension of the droplet size and the pinch-off time difference decreased rapidly (FIG. 3). Above that limit, the situation is more stable where the micelle inside the solution does not affect either the diameter or falling time of droplets.

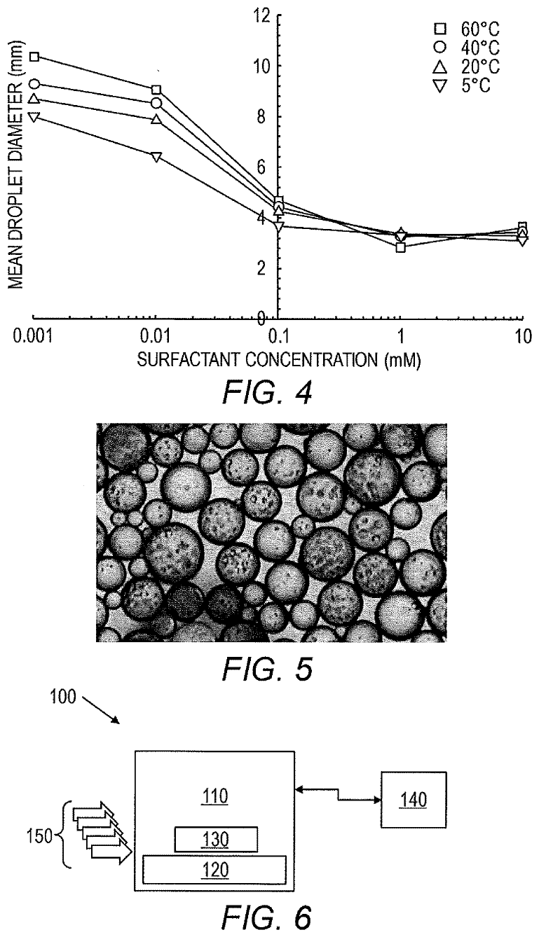

The influence of melting temperature and surfactant concentration on droplet size was also evaluated. This analysis indicated that droplet size increased with temperature at lower concentrations of SPAN® 80 (FIG. 4). At concentrations above the CMC of SPAN® 80, temperature had a reduced influence on droplet size (FIG. 4).

### Example 3: Emulsion Preparation With Stirring

Systems and methods herein can further include the use of a simple magnetic stirrer. Using the same Water-in-Oil emulsion as described in Example 2, which included 10 mM of SPAN® 80, the effect of three different stirring velocities on droplet formation were analyzed, i.e., 50 rpm, 100 rpm and 500 rpm. The higher velocity influenced the Plateau-Rayleigh instability leading to smaller droplets in the microscale size range, and also a more stable emulsion.

### Example 4: Oil-in-Water-in-Oil Emulsion Preparation

The efficiency of phase-change emulsions via melting was further demonstrated using an oil as the dispersed phase. Two different mixtures were prepared. The first was Peanut Oil plus 10 mM of SPAN® 80 (the combination of which was frozen inside a silicon mold) in a continuous phase composed of water with 3 wt % NaCl, 0.2 wt % SDBS (sodium dodecylbenzene sulfonate) and 1 wt % butanol. The second mixture was Peanut Oil with 1 wt % butanol and 10 mM SPAN® 80 (frozen) in 1 wt % TWEEN® 20 (Polyethylene glycol sorbitan monolaurate) blended in water. It was observed that the first mixture did not create an emulsion because it separated into two distinct layers with micelles dispersed in water. In contrast, the second mixture formed a micro-emulsion on the top layer, and its stability was improved by briefly stirring the mixture at the end of the melting phase. The density difference and the small dimension of the oil sphere resulting in the layer of emulsion to float on the water.

After 3 days, the emulsion was observed under a microscope. Surprisingly, the size of the droplets was even smaller than that found in the water-in-oil emulsion.

However, due to long-term instability, some regions presented larger daughter drops, but always in the micrometer size range. Microscopic analysis of each droplet demonstrated that the method described herein can also produce oil-in-water-in-oil emulsions, and by extension can also be used in the preparation of water-in-oil-in-water emulsions. See, e.g., FIG. 5.

1. A method for preparing an emulsion comprising:
(a) providing a multiphase system having an organic phase and an aqueous phase, wherein the organic phase and/or the aqueous phase comprises at least one surfactant;
(b) freezing either the organic phase or the aqueous phase to provide a frozen phase and a liquid phase; and
(c) melting the frozen phase in the liquid phase, thereby preparing an emulsion.

2. The method of claim 1, wherein the multiphase system is an oil-in-water (O/W) emulsion, a water-in-oil (W/O) emulsion, a water-in-oil-in-water (W/O/W) emulsion or an oil-in-water-in-oil (O/W/O) emulsion.

3. The method of claim 1, further comprising the step of applying mechanical energy or thermal energy.

4. The method of claim 1, further comprising adding an excipient to the multiphase system.

5. The method of claim 1, wherein the organic phase comprises a water immiscible solvent.

6. The method of claim 1, wherein the organic phase further includes a co-solvent.

7. The method of claim 6, wherein the co-solvent is a water miscible organic solvent.

8. The method of claim 1, wherein one or both of the organic phase or aqueous phase has an active agent therein.

9. The method of claim 1, wherein the surfactant is nonionic surfactant.

10. The method of claim 9, wherein the nonionic surfactant comprises a polyoxyethylene fatty alcohol ether, polyoxyethylene sorbitan fatty acid ester, polyoxyethylene fatty acid ester, sorbitan ester, glycerol monostearate, polyethylene glycol, polypropylene glycol, cetyl alcohol, cetostearyl alcohol, stearyl alcohol, aryl alkyl polyether alcohol, poloxamer, polaxamine, methylcellulose, hydroxycellulose, hydroxypropylcellulose, hydroxypropylmethylcellulose, noncrystalline cellulose, polysaccharide, polyvinyl alcohol, polyvinylpyrrolidone, or a combination thereof.

11. The method of claim 1, wherein the surfactant in the liquid phase is present in an amount of about the critical micelle concentration of the surfactant.

12. The method of claim 1, wherein the liquid phase has a temperature difference of at least 1° C. from the frozen phase.

13. An emulsion prepared by the method of claim 1.

14. The emulsion of claim 13, wherein the emulsion is comprised by a cosmetic.

15. The emulsion of claim 13, wherein the emulsion is comprised by a pharmaceutical composition.

16. The emulsion of claim 13, wherein the emulsion is comprised by a food.
